(19) United States
(12) Patent Application Publication
(10) Pub. No.: US 2019/0125678 A1
(43) Pub. Date: May 2, 2019

(54) Sustained-Release Oral Dosage Forms for Low Aqueous Solubility Compounds

(71) Applicant: MYLAN, INC., Canonsburg, PA (US)

(72) Inventors: Mummini Aruna Murty, Morgantown, WV (US); John N. Twist, Morgantown, WV (US); Boyong Li, Morgantown, WV (US)

(73) Assignee: MYLAN, INC., Canonsburg, PA (US)

(21) Appl. No.: 15/662,891

(22) PCT Filed: Jan. 29, 2016
(86) PCT No.: PCT/US2016/015647

§ 371 Date: Jul. 28, 2017

Related U.S. Application Data
(60) Provisional application No. 62/110,214, filed on Jan. 30, 2015.

(51) Int. Cl. A61K 9/20; A61K 31/18; A61K 31/4166; A61K 31/437; A61K 31/423; A61K 31/5415; A61K 31/58

(52) U.S. Cl. CPC A61K 9/2031; A61K 31/18; A61K 31/4166; A61K 31/437; A61K 31/423; A61K 9/0053; A61K 31/58; A61K 9/2027; A61K 9/2054; A61K 9/2095; A61K 31/5415

(57) ABSTRACT

Oral dosage forms that contain hydrogel-forming polymers useful for achieving sustained release of API. The formulations disclosed herein may include a hydrogel-forming polymer and a water-insoluble hydrophilic polymer, which acts as a wicking agent to draw water into the formulation. Through that wicking, water combines with the hydrogel-forming polymer to form a hydrogel, thus permitting efficient and sustained release of the API from the hydrogel-forming polymer which may act as a matrix for the API. The formulations disclosed herein are particularly useful for sustained-release of a relatively water-insoluble API, such as tamsulosin, budesonide, and pharmaceutically acceptable salts thereof.

### CROSS-REFERENCE TO RELATED APPLICATIONS

This application claims the benefit of the earlier filing date of U.S. Provisional Patent Application No. 62/110,214 filed on Jan. 30, 2015.

### BACKGROUND OF THE INVENTION

### Field of the Disclosure

The present invention relates generally to oral pharmaceutical dosage forms, and more particularly to sustained-release matrix oral dosage forms containing hydrogels.

### Description of the Background

The advantages of sustained-release products are well known in the pharmaceutical field. One of the greatest benefits of such formulations is the ability to maintain a desired release of the active pharmaceutical ingredient (“API”) over an extended period of time. A sustained-release product reduces the number of administrations required to achieve therapeutic efficacy, and thereby increases patient compliance with a prescribed regimen.

Sustained release of API from oral dosage forms may be achieved by a variety of mechanisms. For example, in some implementations, the API may be encapsulated by a polymeric membrane which permits only slow release of the API from the formulation. Other sustained-release formulations employ a matrix to which the API adheres and from which the API is slowly eluted over time. One commonly employed matrix substrate is a polymeric hydrogel.

A hydrogel is a network of hydrophilic polymers that absorbs quantities of water, yet remains insoluble in aqueous solutions due to crosslinking between polymeric chains. Hydrogels are commonly used in sustained-release drug delivery systems. Often hydrogel-containing, sustained-release formulations combine a hydrogel-forming polymer and a soluble hydrophilic polymer additive. API diffusion out of the gel may be limited by the pore size, as well as the physical and chemical properties of the polymeric hydrogel and how the hydrogel interacts with the API. The hydrophilic polymer allows water to reach the matrix core, swelling the polymeric network, thus promoting release of the API which has been incorporated into the gel during formulation. The release profile of the API in such a hydrogel is thought to be governed by diffusion into and out of the pores, erosion of the gel matrix itself, and the details of how the hydrogel interacts with the API.

Hydrogels may be formulated to achieve diverse API release profiles and permit the formulator to create dosage forms useful for a variety of medical conditions. In modifying the API release profile of the formulation, formulators may promote more rapid release of the API by including water-soluble components in the formulation. Upon exposure of the formulation to aqueous environments (e.g., those found in the stomach and intestines), the water-soluble components dissolve, leaving behind pores in the hydrogel matrix. These pores permit more thorough transfer of API and water into and out of the hydrogel matrix, thus promote more efficient release of the API. To achieve such transport, formulations typically include relatively high levels of such water-soluble additives. When included at sufficiently high concentrations, such components may cause erosion of the polymeric matrix, leading to the disintegration of the formulation. In contrast, if levels of water-soluble components are too low, the voids created by their dissolution will be small, leading to inefficient or highly variable API release from the formulation.

The variability of the distribution of water-soluble components in oral dosage forms creates problems for formulators. The size of the pores (and thus the rate of release of API from the formulation) is significantly impacted by that variable distribution. As such, the API release profile of a formulation may vary significantly, even with formulations having the same overall percentage of components. For formulations containing highly soluble APIs, this problem is attenuated by the rapid dissolution of the API. For formulations containing APIs having low aqueous solubility, such variability in release rates may significantly impact the bioavailability of the API, and thus the therapeutic efficacy of the formulation.

There has been a long-standing need in the pharmaceutical community to improve mechanisms for controlling release profiles of API formulations, particularly those for delivery of APIs having low aqueous-solubility. The present invention addresses this need.

### SUMMARY OF THE INVENTION

The present invention addresses the limitations currently existing within the art and provides sustained-release formulations useful for the delivery of APIs having low aqueous solubility.

The present invention provides oral formulations that contain hydrogel-forming polymers useful for achieving sustained release of an API. The formulations of certain embodiments of the present invention include a hydrogel-forming polymer and a water-insoluble hydrophilic polymer, which acts as a wicking agent. The formulation may also include additional excipients, such as fillers, lubricants, or glidants. The water-insoluble hydrophilic polymer may be present from about 5% to about 20% by weight of the formulation, which may be about 2%, 4%, 6%, 8%, 10%, 12%, 14%, 16%, 18%, 20%, or between any of the aforementioned percentages. The hydrogel-forming polymer may be present from about 20% to about 95% by weight of the formulation, which may be about 20%, 25%, 30%, 35%, 40%, 45%, 50%, 55%, 60%, 65%, 70%, 75%, 80%, 85%, 90%, 95%, or between any of the aforementioned weight percentages. In some embodiments, the hydrogel forming polymer is polyethylene oxide (PEO) and the water-insoluble hydrophilic polymer is crospovidone. In certain embodiments, it has been found that incorporating PEO at about 90% by weight is particularly useful. This formulation is particularly useful for sustained-release of a relatively water-insoluble API, however it can be used to deliver any API or bioactive agent that can be incorporated into the hydrogel.

One object of the present invention is a sustained-release solid pharmaceutical dosage form that includes a hydrogel-forming polymer, a hydrophilic water-insoluble polymer, and an active pharmaceutical ingredient, where the hydrophilic water-insoluble polymer is present throughout the oral dosage form and where the hydrophilic water-insoluble polymer is capable of acting as a wicking agent. In some embodiments, the hydrophilic water-insoluble polymer is substantially uniformly present throughout the oral dosage form. The hydrogel-forming polymer may be present at a concentration from about 20% to about 95% by weight of the formulation, which may be about 20%, 25%, 30%, 35%, 40%, 45%, 50%, 55%, 60%, 65%, 70%, 75%, 80%, 85%, 90%, 95%, or between any of the aforementioned weight percentages. The hydrogel-forming polymer may, for example, be polyvinyl pyrrolidone, hydroxypropyl methylcellulose, hydroxypropyl cellulose, polyethylene glycol, hydroxyethyl cellulose, polyethyelene oxide, carbomer, polyvinyl alcohol, or mixtures thereof. The hydrophilic water-insoluble polymer may be present at a concentration from about 2% to about 20% by weight, which may be about 2%, 4%, 6%, 8%, 10%, 12%, 14%, 16%, 18%, 20%, or between any of the aforementioned weight percentages. The hydrophilic water-insoluble polymer may, for example, be crospovidone, croscarmellose sodium, sodium starch glycolate, carboxymethylcellulose sodium, starch and derivatives of starch, or mixtures thereof.

Such sustained-release solid pharmaceutical dosage forms of the present invention are particularly effective at delivering active pharmaceutical ingredients having low aqueous solubility. Examples include tamsulosin, budesonide, zolpidem, phenytoin, meloxicam, and zonisamide. The sustained-release solid pharmaceutical dosage form may also include additional components, such as binders, fillers, glidants, lubricants, preservatives, coloring agents, flavoring agents, or mixtures thereof to facility formulation of the final dosage form.

A further object of the present invention includes a method of formulating a sustained-release solid dosage form that includes the steps of combining a hydrogel-forming polymer with a first mass of hydrophilic water-insoluble polymer to form a pre-blend, mixing the pre-blend, dissolving an active pharmaceutical ingredient in a solvent to form a solution, granulating said pre-blend using said solution to form a plurality of granules, adding a second mass of hydrophilic water-insoluble polymer to form a final blend, and compressing said final blend into a tablet. The first mass of hydrophilic water-insoluble polymer is about 25% to about 75% of the total amount of hydrophilic water-insoluble polymer to be added to the sustained-release solid dosage form. For example, about 25%, 27%, 29%, 31%, 33%, 35%, 37%, 39%, 41%, 43%, 45%, 47%, 49%, 51%, 53%, 55%, 57%, 59%, 61%, 63%, 65%, 67%, 69%, 71%, 73%, 75%, or any percentages in between the aforementioned weight percentages may be added at this step. The solvent may be, for example, isopropyl alcohol, ethanol, methanol, water, acetone, and miscible mixtures thereof. The sustained-release solid pharmaceutical dosage form made by these methods may include the components as disclosed above.

A further object of the present invention is a sustained-release solid pharmaceutical oral dosage form that includes about 90% polyethylene oxide, about 10% crospovidone, and about 0.4 mg of tamsulosin, where crospovidone is present throughout the oral dosage form and where the crospovidone is capable of acting as a wicking agent for water. In some embodiments, the crospovidone is substantially uniformly present throughout the oral dosage form.

A further object of the present invention is a sustained-release solid pharmaceutical oral dosage form that includes about 90% polyethylene oxide, about 10% crospovidone, and about 3 mg or about 9 mg of budesonide, where crospovidone is present throughout the oral dosage form and where the crospovidone is capable of acting as a wicking agent for water. In some embodiments, the crospovidone is substantially uniformly present throughout the oral dosage form.

### BRIEF DESCRIPTION OF THE DRAWINGS

Further aspects of the present disclosure together with additional features contributing thereto and advantages accruing there from will be apparent from the following description of embodiments of the disclosure, which are shown in the accompanying drawing FIGURES wherein:

FIG. 1 shows a release profile for embodiments of the present invention that contain tamsulosin as API.

### DETAILED DESCRIPTION OF THE INVENTION

It is to be understood that the figures and descriptions of the present invention have been simplified to illustrate elements that are relevant for a clear understanding of the invention. The detailed description will be provided herein below with reference to the attached drawing.

The present invention provides a novel hydrogel-based, sustained-release oral dosage formulation of an API. The sustained-release dosage forms of the present invention may be useful for administration of API with low aqueous solubility, API with a fast mechanism of action, or any other API for which a consistent sustained-release profile is desired.

The sustained-release dosage forms of the present invention may be prepared as a combination of polymers including one or a mixture of hydrogel-forming polymers and a water-insoluble hydrophilic polymer. The formulations of the present invention may also include one or more excipients.

In the context of the present invention and as described further herein below, the water-insoluble hydrophilic polymer may act as a wicking agent to draw water into the formulation, thereby promoting formation of a hydrogel upon ingestion of the dosage form after ingestion by the patient. In some embodiments, crospovidone serves as the water-insoluble hydrophilic polymer. The water-insoluble hydrophilic polymer may also aid in the controlled release of the API from the hydrogel. The various embodiments of the present invention may provide numerous advantages over prior art, including inclusion of a reduced amount of water-insoluble polymer used during formulation and improved control over the API release profile, when compared to prior art formulations.

Generally, pharmaceutical formulations may employ hydrogels to achieve sustained release of the API. The hydrogel may act as a matrix in which API is uniformly distributed during formulation, and from which API slowly elutes over time after ingestion. In those formulations, the release profile of the API from the dosage form may be shaped by the specific composition of the hydrogel, which may erode slowly as the dosage form passes through the gastrointestinal tract. The water solubility of the components of a hydrogel-based formulation may further impact the structural stability of the formulation, as well as the release rate of the API.

The present invention improves upon this art by providing a novel structure and mechanism for achieving sustained release from oral dosage forms. In some embodiments of the present invention, a hydrogel-forming polymer is included as a main component of the formulation. In some particularly useful embodiments, the hydrogel-forming component is a hydrophilic polymer. Generally, any polymer which is capable of forming a hydrogel upon exposure to aqueous environments may be used within the context of the present invention. Examples of hydrogel-forming polymers useful within the context of the present invention include polyvinyl pyrrolidone (povidone), hydroxypropyl methylcellulose, hydroxypropyl cellulose, polyethylene glycol (PEG), hydroxyethyl cellulose, polyethyelene oxide (PEO), carbomer, polyvinyl alcohol, and mixtures thereof. One of skill in the art will recognize other well-known hydrogel-forming substances that may be used within the context of the present invention.

Within the context of embodiments of the present invention, the hydrogel-forming polymer may be included in the formulation at a wide range of concentrations. Primarily, the hydrogel-forming polymer may be included at sufficient levels to achieve formation of a hydrogel upon exposure to aqueous environments. In specific embodiments of the present invention, the hydrogel-forming polymer may be included at concentrations from about 10% to about 95% by weight, which may be about 10%, 15%, 20%, 25%, 30%, 35%, 40%, 45%, 50%, 55%, 60%, 65%, 70%, 75%, 80%, 85%, 90%, 95%, or in between any of the aforementioned weight percentages. In some embodiments, concentration of the hydrogel-forming polymer may be between about 20% to about 95%, which may be about 20%, 25%, 30%, 35%, 40%, 45%, 50%, 55%, 60%, 65%, 70%, 75%, 80%, 85%, 90%, 95%, or in between any of the aforementioned weight percentages. In certain embodiments of the present invention, polyethylene oxide is used as the hydrogel-forming polymer. In particularly useful embodiments, polyethylene oxide is included at about 90% by weight of the formulation as the hydrogel-forming polymer.

The formulations of the present invention may also include a water-insoluble hydrophilic polymer capable of acting as a wicking agent. The water-insoluble hydrophilic polymer may be distributed throughout the dosage form, including throughout the hydrogel-forming polymer. In some embodiments, the water-insoluble hydrophilic polymer is substantially uniformly present throughout the oral dosage form. While not wishing to be bound to theory, it is believed that the water-insoluble hydrophilic polymer acts as a wicking agent, drawing water from the gastrointestinal tract into the unit dosage form. As this component is water insoluble, the water-insoluble hydrophilic polymer remains in place allowing water to be drawn into the formulation over extended periods of time. Also, because the water-insoluble polymer remains does not erode, but rather remains in place, only low levels of the water-insoluble hydrophilic component are needed to be included in the formulation to establish this functionality. Once drawn into the formulation by the water-insoluble hydrophilic polymer, water combines with hydrogel-forming polymer to form a hydrogel.

Following formation of the hydrogel, the wicking agent embodied by the water-insoluble hydrophilic polymer also permits the efficient elution of API from the hydrogel matrix. It is believed that the efficacy of those transport mechanisms is maintained as the water-insoluble hydrophilic polymer remains in place as the formulation passes through the gastrointestinal tract. Further, the structural integrity of the unit dosage form may be maintained through the delivery of API as the dosage form passes through the gastrointestinal tract. This attribute may further provide for reliable and consistent release of API from the formulations of the present invention.

In some embodiments, the water-insoluble hydrophilic polymer is crospovidone. Other examples of pharmaceutically acceptable water-insoluble polymers useful within the context of the present invention are croscarmellose sodium and sodium starch glycolate, carboxymethylcellulose sodium starch and derivatives of starch, and mixtures thereof. Functionally, any material that is capable of wicking water into the hydrogel without substantially dissolving may be used within the context of the present invention.

Advantageously, the formulations of the present invention may be prepared using low levels of water-insoluble polymer. In contrast, prior art oral dosage forms, for example, those disclosed in U.S. Pat. No. 6,699,503, which is hereby incorporated by reference for the disclosure relating to dosage form formulation, commonly incorporate a soluble hydrophilic polymer. To achieve consistent release from such formulations (through consistent penetration of the water into the formulation), the percentage of that the water-soluble polymer may typically be present at higher weight percentages, for example from about 14% to about 64% by weight, which may be about 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, 62%, 64% or between any of the aforementioned weight percentages. In contrast, some embodiments of the present invention incorporate the water-insoluble hydrophilic polymer from about 2% to about 20% by weight, which may be about 2%, 4%, 6%, 8%, 10%, 12%, 14%, 16%, 18%, 20%, or between any of the aforementioned percentages.

The present invention is particularly useful for APIs having low aqueous solubility. The pharmaceutical dosage forms of the present invention may achieve a more stable release profile of an API with low aqueous solubility. In addition to being particularly useful with API having low aqueous solubility, the present invention may also be employed for sustained-release of any API or molecule that can be incorporated into a hydrogel-forming polymer.

Examples of APIs useful within the context of the methods and dosage forms of the present invention include tamsulosin, budesonide, zolpidem, phenytoin, meloxicam, and zonisamide. Formulations of tamsulosin as the API are particularly useful within the context of the present invention. The API may be present at a concentration which will achieve the desired physiological effect. For example, tamsulosin may be present at 0.4 mg/dosage form. As a further example, budesonide may be present at 3-9 mg/dosage form. The API may be present as a pharmaceutically acceptable salt. The diversity of available pharmaceutically acceptable salts will be dictated by the specific API being used. For example, tamsulosin may be present as tamsulosin HCl.

The dosage forms of the present invention may be prepared by methods well known in the art. In general, the formulations of the present invention are formulated such that a water-insoluble hydrophilic polymer is present throughout the formulation. In some embodiments, the water-insoluble hydrophilic polymer is substantially uniformly present throughout the oral dosage form. In this manner, the water-insoluble hydrophilic polymer may efficiently draw water into a substantial portion of the dosage form, thus achieving both relatively uniform hydrogel formation as well providing an extensive network of channels for release of the API from the formulation. In some embodiments of the present invention, dosage forms may be generated through wet granulation, though one of skill in the art would recognize a diversity of additional methods useful for preparing a formulation, for example, dry granulation.

According to the present invention, the hydrogel-forming polymer and the hydrophilic water-insoluble polymer may be combined to form a pre-blend. In certain embodiments of the invention, only a portion of the hydrophilic water-insoluble polymer may be added initially, for example, about 25% to about 75% of the total amount of hydrophilic water-insoluble polymer. For example, about 25%, 27%, 29%, 31%, 33%, 35%, 37%, 39%, 41%, 43%, 45%, 47%, 49%, 51%, 53%, 55%, 57%, 59%, 61%, 63%, 65%, 67%, 69%, 71%, 73%, 75%, or any percentages in between the aforementioned weight percentages may be added at this step.

The API may then be separately dissolved in an appropriate solvent. Examples of useful solvents include isopropyl alcohol, ethanol, methanol, water, acetone, and miscible combinations thereof. The specific choice of solvent will be, in part, dictated by the particular API used in the formulation. A particularly useful solvent for tamsulosin is isopropyl alcohol. After the API is dissolved in solvent to form a solution, that solution may be added to the mixture of hydrogel-forming polymer and hydrophilic water-insoluble polymer. The mixture may then be granulated, and the granules dried and milled to a substantially uniform size. While not wishing to be bound by theory, the use of a water-insoluble polymer in the formulation of the dosage forms of the present invention may act to further improve the preparation process by reducing clumping and adhesion of components to equipment during wet granulation and blend-mill-blend formulation processes.

Additional water-insoluble hydrophilic polymer and any desired excipients (e.g., binders, fillers, glidants, lubricants, preservatives, coloring agents, flavoring agents) may then be added to the granules composition to form a final blend. This final blend may then be formulated into a final dosage form. For example, the blend may be compressed into tablets or the granules may be used as a fill for capsules.

As noted above, the formulations of the present invention may also include further excipients to achieve the desired physical properties for the final dosage form. Examples of useful binders include saccharides and their derivatives such as sucrose, lactose, starches, cellulose, and sugar alcohols, gelatins. Examples of useful fillers include lactose, sucrose, magnesium stearate, glucose, mannitol, sorbitol, calcium phosphate, and calcium carbonate. Examples of useful glidants/lubricants include talc, silicon dioxide (colloidal), sodium stearyl fumarate, magnesium stearate, sodium lauryl sulfate, calcium stearate, magnesium lauryl sulfate, potassium benzoate, sodium benzoate, zinc stearate, and mixtures thereof. Examples of useful preservatives include butylated hydroxytoluene, vitamin A, vitamin E, vitamin C, retinyl palmitate, selenium, methyl paraben, propyl paraben, and derivatives thereof. The above examples of excipients are not limited to just those mentioned but are simply examples of excipients that could be useful in the present invention. One skilled in the art would recognize other similar excipients that would be useful in the preparation of the invention. The mixture may then be processed into a suitable oral dosage form, for example, compressed into a tablet.

Nothing in the above description is meant to limit the present invention to any specific materials, geometry, or orientation of elements. Many substitutions and variations are contemplated within the scope of the present invention, will be apparent to those skilled in the art, and may be achieved without undue experimentation. The embodiments described herein were presented by way of example only and should not be used to limit the scope of the invention.

Although the invention has been described in terms of particular embodiments in an application, one of ordinary skill in the art, in light of the teachings herein, can generate additional embodiments and modifications without departing from, or exceeding the scope of, the claimed invention. Accordingly, it is understood that the drawings and the descriptions herein are proffered only to facilitate comprehension of the invention and should not be construed to limit the scope thereof.

The following examples are provided for illustrative purposes only and do not limit the scope of the invention in any way.

### EXAMPLES

### Examples 1-4: Pharmaceutical Compositions of Tamsulosin

The compositions of four representative examples with tamsulosin as the API (Examples 1-4) are provided in Table 1. Values listed are amounts in milligrams per unit dosage form.

TABLE 1

| Component | Example 1 | Example 2 | Example 3 | Example 4 |
|---|---|---|---|---|
| Tamsulosin HCl | 0.4 | 0.4 | 0.4 | 0.4 |
| Crospovidone | 12.25 | 19.45 | 24.30 | 29.17 |
| Polyethylene | 223.78 | 23.68 | 218.80 | 213.95 |
| Oxide | | | | |
| Talc | 6.125 | — | — | — |
| Sodium Stearyl | 1.225 | 1.225 | 1.225 | 1.225 |
| Fumarate | | | | |
| Colloidal Silicon | 1.225 | — | — | — |
| Dioxide | | | | |
| Butylated | — | 0.245 | 0.245 | 0.245 |
| Hydroxytoluene | | | | |

### Example 5: Release Profiles for Examples 1-4

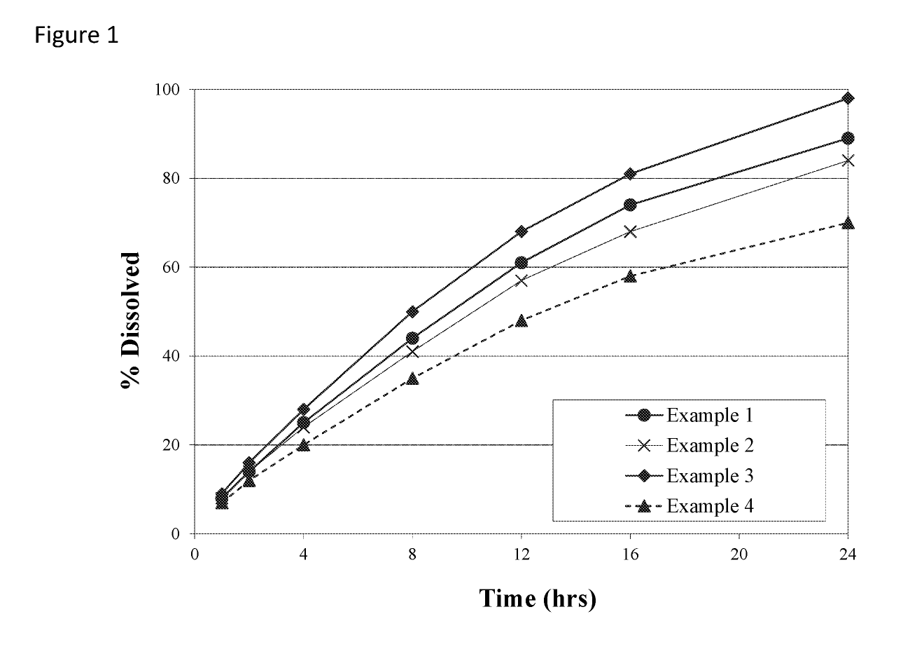

The release profile of each of the above examples 1-4 was tested using the paddle method in pH 6.8 buffer. The results of that test are shown in FIG. 1, as the percent of tamsulosin HCl released from the formulation as a function of time. As is seen in FIG. 1, certain embodiments of the present invention may effectively achieve sustained release of the API over an extensive period of time.

### Examples 6 and 7: Pharmaceutical Compositions of Budesonide

The compositions of two representative examples with budesonide as the API 0 are provided in Table 2. Values listed are amounts in milligrams per unit dosage form.

TABLE 2

| Component | Example 6 | Example 7 |
|---|---|---|
| Budesonide | 3 | 9 |
| Crospovidone | 19.45 | 19.45 |
| Polyethylene | 23.68 | 23.68 |
| Oxide | | |
| Talc | — | — |
| Sodium Stearyl | 1.225 | 1.225 |
| Fumarate | | |
| Colloidal Silicon | — | — |
| Dioxide | | |
| Butylated | 0.245 | 0.245 |
| Hydroxytoluene | | |

We claim:

1. A sustained-release solid oral dosage form, comprising:
a. a hydrogel-forming polymer;
b. a hydrophilic water-insoluble polymer; and
c. an active pharmaceutical ingredient.

2. The sustained-release solid oral dosage form of claim 1, wherein the hydrophilic water-insoluble polymer is present throughout the sustained-release oral dosage form.

3. The sustained-release solid oral dosage form of claim 1, wherein the hydrophilic water-insoluble polymer is capable of acting as a wicking agent for water when the sustained-release solid oral dosage form is placed in an aqueous environment, such that water is drawn into the sustained-release solid oral dosage form by the water-insoluble polymer.

4. The sustained-release solid oral dosage form of claim 1, wherein said hydrogel-forming polymer is present at a concentration from about 20% to about 95% by weight of the sustained-release oral dosage form.

5. The sustained-release solid oral dosage form of claim 1, wherein said hydrogel-forming polymer is selected from the group consisting of polyvinyl pyrrolidone, hydroxypropyl methylcellulose, hydroxypropyl cellulose, polyethylene glycol, hydroxyethyl cellulose, polyethyelene oxide, carbomer, polyvinyl alcohol, and mixtures thereof.

6. The sustained-release solid oral dosage form of claim 1, wherein said hydrophilic water-insoluble polymer is present at a concentration from about 2% to about 20% by weight of the sustained-release oral dosage form.

7. The sustained-release solid oral dosage form of claim 1, wherein said hydrophilic water-insoluble polymer is selected from the group consisting of crospovidone, croscarmellose sodium, sodium starch glycolate, carboxymethylcellulose sodium, starch and derivatives of starch, and mixtures thereof.

8. The sustained-release solid oral dosage form of claim 1, wherein said active pharmaceutical ingredient has low aqueous solubility.

9. The sustained-release solid oral dosage form of claim 1, wherein the active pharmaceutical ingredient is selected from the group consisting of tamsulosin, budesonide, zolpidem, phenytoin, meloxicam, and zonisamide.

10. The sustained-release solid oral dosage form of claim 1, further comprising one or more pharmaceutically acceptable excipients.

11. The sustained-release solid oral dosage form of claim 1, wherein the pharmaceutically acceptable excipient is selected from the group consisting of binders, fillers, glidants, lubricants, preservatives, coloring agents, flavoring agents, and mixtures thereof.

12. A method of formulating a sustained-release solid oral dosage form, comprising the steps of:
a. combining a hydrogel-forming polymer with a first amount of hydrophilic water-insoluble polymer to form a pre-blend;
b. mixing said pre-blend;
c. dissolving an active pharmaceutical ingredient in a solvent to form a solution;
d. granulating said pre-blend using said solution to form a plurality of granules;
e. adding a second amount of hydrophilic water-insoluble polymer to form a final blend; and
f. compressing said final blend into a tablet.

13. The method of claim 12, wherein said adding step further comprises adding an excipient.

14. The method of claim 13, wherein said excipient is selected from the group consisting of binders, fillers, glidants, lubricants, preservatives, coloring agents, and flavoring agents.

15. The method of claim 12, wherein said first amount of hydrophilic water-insoluble polymer is about 25% to about 75% of the total amount of hydrophilic water-insoluble polymer to be added to the sustained-release solid oral dosage form.

16. The method of claim 12, wherein said solvent is selected from the group consisting of isopropyl alcohol, ethanol, methanol, water, acetone, and miscible mixtures thereof.

17. The method of claim 12, wherein said hydrogel-forming polymer is selected from the group consisting of polyvinyl pyrrolidone, hydroxypropyl methylcellulose, hydroxypropyl cellulose, polyethylene glycol, hydroxyethyl cellulose, polyethyelene oxide, carbomer, polyvinyl alcohol, and mixtures thereof.

18. The method of claim 1, wherein said hydrogel-forming polymer is present at a concentration from about 20% to about 90% by weight of the sustained-release solid oral dosage form.

19. The method of claim 12, wherein said hydrophilic water-insoluble polymer is selected from the group consisting of crospovidone, ethyl cellulose, cellulose acetate, cellulose phthalate, starch, alginic acid, polyvinyl acetate, acrylate-methacrylate copolymers, acrylate polymers, and mixtures thereof.

20. The method of claim 12, wherein the first amount and second amount of said hydrophilic water-insoluble polymer together are present at a concentration from about 2% to about 20% by weight of the sustained-release solid oral dosage form.

21. The method of claim 12, wherein the active pharmaceutical ingredient is selected from the group consisting of tamsulosin, budesonide, zolpidem, phenytoin, meloxicam, and zonisamide.

22. A sustained-release solid oral dosage form, comprising:
a. about 90% polyethylene oxide by weight of the sustained-release oral dosage form;
b. about 10% crospovidone by weight of the sustained-release oral dosage form; and
c. about 0.4 mg of tamsulosin.

23. The sustained-release solid oral dosage form of claim 22, further comprising an excipient selected from the group consisting of talc, sodium stearyl fumarate, colloidal silicon dioxide, butylated hydroxytoluene, and mixtures thereof.

24. The sustained-release solid oral dosage form of claim 22, wherein the crospovidone is present throughout the sustained-release solid pharmaceutical oral dosage form, and wherein the crospovidone acts as a wicking agent for water when the sustained-release solid pharmaceutical oral dosage form is in an aqueous environment.
